(19) United States
(12) Patent Application Publication
(10) Pub. No.: US 2025/0117938 A1
(43) Pub. Date: Apr. 10, 2025

(54) HEMOGLOBIN ANALYSIS METHOD AND SYSTEM BASED ON A MICROSCOPICALLY MAGNIFIED DIGITAL IMAGE

(71) Applicant: Shenzhen Anlv Medical Technology Co., Ltd, Shenzhen (CN)

(72) Inventors: Zhiping WANG, Shenzhen (CN); Yahui LIU, Shenzhen (CN); Xiangfei FANG, Shenzhen (CN); Chunshu WANG, Shenzhen (CN)

(73) Assignee: Shenzhen Anlv Medical Technology Co., Ltd, Shenzhen (CN)

(21) Appl. No.: 18/982,748

(22) Filed: Dec. 16, 2024

Related U.S. Application Data
(63) Continuation of application No. PCT/CN2022/124646, filed on Oct. 11, 2022.

(30) Foreign Application Priority Data
Jun. 17, 2022 (CN) 202210684094.3

(51) Int. Cl. G06T 7/00

(52) U.S. Cl. CPC G06T 7/0012; G06T 2207/10024; G06T 2207/10056; G06T 2207/30024

(57) ABSTRACT

A hemoglobin analysis method based on a microscopically magnified digital image. On the basis of the first absorbance of target cells α1=log(the mean gray value Gb of the blank area/the mean gray value Gc of the target cell area), target corpuscular hemoglobin CH=(STC/KHGB)×lg(Gb/Gc) is acquired. The target corpuscular hemoglobin CH=the first absorbance α1×the target cell area STC×the first hemoglobin correction coefficient CHB1. By using the Beer-Lambert law, the hemoglobin content of a single target cell can be accurately acquired.

### CROSS-REFERENCE TO RELATED APPLICATIONS

This application is a continuation of International (PCT) Patent Application No. PCT/CN2022/124646, filed on Oct. 11, 2022, which claims the priority of Chinese Patent Application No.202210684094.3, filed on Jun. 17, 2022, both of which are herein incorporated by reference in their entirety.

### TECHNICAL FIELD

The present disclosure relates to the technical field of obtaining the characteristics and parameters of various components in a target sample based on microscopically magnified digital images of cell suspensions, particularly relating to the technical field of analyzing specific components within cells using digital images, and specifically relates to the technical field of analyzing corpuscular hemoglobin using microscopically magnified digital images of cell suspensions.

### BACKGROUND

The Beer-Lambert law is one of the commonly used principles for determining the content of various substances. As shown in FIG. 1, its physical significance is that when a beam of parallel monochromatic light passes perpendicularly through a uniform non-scattering absorbing substance, the absorbance A is directly proportional to the concentration c of the absorbing substance and the thickness b of the absorbing layer, and inversely related to the transmittance T. The mathematical expression of the Beer-Lambert law is: A=log(1/T)=Kbc; A represents the absorbance; T represents the transmittance, which is equal to the intensity of the transmitted light Iₜ divided by the intensity of the incident light I₀; K represents the molar absorption coefficient, which is related to the nature of the absorbing substance and the wavelength λ of the incident light; c represents the concentration of the absorbing substance; b represents the thickness of the absorption layer, which is often replaced by 1 with the same meaning. The conditions for the Beer-Lambert law to be applicable include: (1) the incident light is parallel monochromatic and perpendicularly incident; (2) the absorbing substance is a uniform, non-scattering system; (3) there is no interaction between the absorbing particles; and (4) the interaction between the radiation and substance only allows light absorption.

In the related art, the principle of a hematology analyzer detecting the corpuscular hemoglobin (HGB) per liter of a blood cell sample is shown in FIG. 2. After adding a hemolytic agent to the blood, the target cells release hemoglobin, which combines with the hemolytic agent to form hemoglobin derivatives, i.e., Hb derivatives. These Hb derivatives are uniformly dispersed in the sample, giving the sample uniform non-scattering characteristics. Therefore, the absorption characteristics of Hb derivatives at specific wavelengths (530-550 nm) can be utilized, i.e., the Beer-Lambert law can be used to measure absorbance. By measuring the change in the amount of absorbed light, the content of the Hb derivatives in the liquid can be determined. The content of the Hb derivatives corresponds to the HGB content. Thus, the HGB content can be measured using the above method. The usual unit for HGB content is g/L, which represents the mass of hemoglobin per unit volume.

Blood is composed of blood cells (red blood cells, white blood cells, and platelets) and plasma. When blood is not treated with anticoagulants after being drawn (or be centrifuged), it will naturally coagulate, resulting in the separation of a light-yellow transparent liquid on the top, which is serum, a white solid layer in the middle, which is white blood cells and platelets, and a red solid layer at the bottom, which is the target cells. Hemoglobin is normally encapsulated within the cell membrane. Since most blood cells in a whole blood sample are encapsulated by cell membranes, the blood sample will naturally stratify when placed in a conventional test tube-like container and does not exhibit uniform non-scattering characteristics. Therefore, in the related art, the most used method for detecting the corpuscular hemoglobin (HGB) per liter of blood cell sample with a hematology analyzer is the HiCN assay. The HiCN assay, or hemoglobin cyanide (HiCN) spectrophotometric assay, is a reference recommended by the World Health Organization and the International Committee for Standardization in Hematology. Its assay results serve as a traceability standard for other hemoglobin assays. The principle of the cyanmethemoglobin spectrophotometry is that the ferrous ions (Fe²⁺) in hemoglobin (except sulfhemoglobin) are oxidized into ferric ions (Fe³⁺) by potassium ferrocyanide, converting hemoglobin into methemoglobin. Methemoglobin combines with cyanide ions (CN⁻) to form stable cyanide methemoglobin (HiCN). When detected with a spectrophotometer, cyanmethemoglobin has a broad absorption peak at a wavelength of 540 nm, and its absorbance at 540 nm is proportional to its concentration in the solution. The HiCN assay requires hemolysis first, allowing hemoglobin to combine with the hemolytic agent to form hemoglobin derivatives, giving the sample uniform non-scattering characteristics before the Beer-Lambert law can be used. In the process of measuring hemoglobin as described above, a hemolytic agent is required to dissolve hemoglobin from the target cells. The hemolysis process destroys the overall structure of the cells. Therefore, in blood analysis, counting the blood cells first and then performing hemolysis are usually considered. The whole blood analysis process is constrained by this requirement and must be performed in a specific sequence. Additionally, the introduction of a hemolytic agent in the intermediate process makes the entire operation more complex, reducing overall efficiency.

In the related art, since hemolysis is required for measuring corpuscular hemoglobin (HGB), the opportunity to accurately obtain the corpuscular hemoglobin of a single red blood cell is lost. Therefore, traditional hematology analyzers can only output a quantitative analysis result of the corpuscular hemoglobin (HGB) of the sample, without accurately delving into the level of a single red blood cell or deeper cellular levels.

However, in actual clinical applications and research, the size of each red blood cell, the corpuscular hemoglobin of each red blood cell, and their distribution patterns and characteristics all represent corresponding physiological or pathological significances.

### Glossary

WBC is the abbreviation of “white blood cell”. In the field of blood analyzers, WBC refers to white blood cell concentration, with the unit being “cells/L”.

RBC is the abbreviation of “red blood cell”. In the field of blood analyzers, RBC refers to red blood cell concentration, with the unit being “cells/L”.

HCT is the abbreviation of “hematocrit”, called PCV. In the field of blood analyzers, HCT means the volume ratio of red blood cells to whole blood after anticoagulation, with the unit being %.

CV is the abbreviation of “corpuscular volume”, with the unit being “fL”.

MCV is the abbreviation of “mean corpuscular volume”. In the field of blood analyzers, MCV represents the mean volume of all red blood cells, and the unit is “femtoliter (fL)”.

HGB is the abbreviation of “hemoglobin”. In the field of blood analyzers, HGB means the corpuscular hemoglobin per unit volume of blood, i.e., the hemoglobin concentration, and the unit is “g/L”.

CH is the abbreviation of “corpuscular hemoglobin”. In the field of blood analyzers, CH means the corpuscular hemoglobin of a single red blood cell, and the unit is “pg”.

MCH is the abbreviation of “mean corpuscular hemoglobin”. In the field of blood analyzers, MCH means the mean corpuscular hemoglobin of a single red blood cell, and the unit is “picogram (pg)”.

MCHC is the abbreviation of “mean corpuscular hemoglobin concentration”. In the field of blood analyzers, MCHC means the mean corpuscular hemoglobin concentration per unit volume of red blood cells, and the unit is “g/L”.

In the calculation process of traditional blood analyzers, MCHC=HGB÷RBC÷MCV; MCHC=MCH÷MCV=HGB÷RBC÷MCV, MCH=HGB÷RBC.

### SUMMARY

A technical solution to solve the above problem is a hemoglobin analysis method based on a microscopically magnified digital image. The microscopically magnified digital image is obtained based on blood cells laid in a monolayer in a suspension; and the method includes the following steps: (6A): identifying multiple target cells in the microscopically magnified digital image; (6B): selecting a target image corresponding to each target cell in the microscopically magnified digital image; the target image including a target cell area and a blank area; (6C): based on a mean grayscale value of the target cell area in the target image and a mean grayscale value of the blank area in the target image, calculating a first absorbance of the target cell using a formula: the first absorbance of the target cell=log (the mean grayscale value of the blank area in the target image/the mean grayscale value of the target cell area in the target image); (6I): obtaining a first hemoglobin absorption coefficient; (6J): calculating a corpuscular hemoglobin per unit area of the target cell using a formula: the corpuscular hemoglobin per unit area of the target cell=the first absorbance of the target cell/the first hemoglobin absorption coefficient; (6K): obtaining each target cell area in the microscopically magnified digital image; and (6L): calculating a corpuscular hemoglobin of each target cell using a formula: the corpuscular hemoglobin of each target cell=each target cell area×the corpuscular hemoglobin per unit area of the target cell, or the corpuscular hemoglobin of each target cell=each target cell area/the first hemoglobin absorption coefficient× log (the mean grayscale value of the blank area in the target image/the mean grayscale value of the target cell area in the target image). In some embodiments, the step (6I) of obtaining the first hemoglobin absorption coefficient includes the following steps: (6IA1): taking an amount of blood cell sample to be analyzed, and obtaining a corpuscular hemoglobin and a red blood cell concentration per liter of the blood cell sample using a hemoglobin analyzer; (6IA2): taking a same amount of the blood cell sample to be analyzed as at step (6IA1), obtaining a cell suspension by pre-treating, injecting the cell suspension into an imaging target area; making a monolayer of blood cells in the suspension, and obtaining a microscopically magnified digital image of the monolayer of blood cells in the suspension; (6IA3): selecting a target image corresponding to each target cell in the microscopically magnified digital image obtained at step (6IA2), the corresponding target image including a target cell area and a blank area; (6IA4): based on a mean grayscale value of each target cell area and a mean grayscale value of the blank area in each target image obtained at step (6IA3), calculating a first absorbance of each target cell using a formula: the first absorbance of each target cell=log (the mean grayscale value of the blank area/the mean grayscale value of each target cell area); (6IA5): obtaining a mean value of the first absorbance based on the first absorbance of each target cell obtained at step (6IA4); (6IA6): obtaining each target cell area and calculating a mean area of all target cells; and (6IA7): calculating the first hemoglobin absorption coefficient using a formula: the first hemoglobin absorption coefficient=the mean value of the first absorbance÷the corpuscular hemoglobin per unit area of the target cell; or the first hemoglobin absorption coefficient=the mean value of the first absorbance÷(the corpuscular hemoglobin per liter of blood cell sample÷the red blood cell concentration÷the mean area of all target cells)=the mean value of the first absorbance×the red blood cell concentration×the mean area of all target cells÷the corpuscular hemoglobin per liter of blood cell sample.

A hemoglobin analysis method based on a microscopically magnified digital image, wherein the microscopically magnified digital image is obtained based on blood cells laid in a monolayer in a suspension; and the method includes the following steps: (6A): identifying multiple target cells in the microscopically magnified digital image; (6B): selecting a target image corresponding to each target cell in the microscopically magnified digital image; the target image including a target cell area and a blank area; (6C): based on a mean grayscale value of the target cell area in the target image and a mean grayscale value of the blank area in the target image, calculating a first absorbance of the target cell using a formula: the first absorbance of the target cell=log (the mean grayscale value of the blank area in the target image/the mean grayscale value of the target cell area in the target image); (7I): obtaining a known first corpuscular hemoglobin correction coefficient; (7K): obtaining each target cell area in the microscopically magnified digital image; (7J): calculating a target cell corpuscular hemoglobin using a formula: the target cell corpuscular hemoglobin=the first absorbance×each target cell area×the first corpuscular hemoglobin correction coefficient. In some embodiments, the step (7I) of obtaining the first corpuscular hemoglobin correction coefficient includes the following steps: (7IA1): taking an amount of blood cell sample to be analyzed, and obtaining a corpuscular hemoglobin and a red blood cell concentration per liter of the blood cell sample using a hemoglobin analyzer; (7IA2): taking a same amount of the blood cell sample to be analyzed as at step (7IA1), obtaining a cell suspension by pre-treating, injecting the cell suspension into an imaging target area; making a monolayer of blood cells in the suspension, and obtaining a microscopically magnified digital image of the monolayer of blood cells in the suspension; (7IA3): selecting a target image corresponding to each target cell in the microscopically magnified digital image obtained at step (7IA2), the corresponding target image including a target cell area and a blank area; (7IA4): based on a mean grayscale value of each target cell area and a mean grayscale value of the blank area in each target image obtained at step (7IA3), calculating a first absorbance of each target cell using a formula: the first absorbance of each target cell=log (the mean grayscale value of the blank area in each target image/the mean grayscale value of each target cell area); (7IA5): obtaining a mean value of the first absorbance based on the first absorbance of each target cell obtained at step (7IA4); (7IA6): obtaining each target cell area and calculating a mean area of all target cells; and (7IA7): calculating the first corpuscular hemoglobin correction coefficient using a formula: the first corpuscular hemoglobin correction coefficient=the corpuscular hemoglobin per liter of blood cell sample÷the red blood cell concentration÷the mean value of the first absorbance÷the mean area of all target cells.

A hemoglobin analysis method based on a microscopically magnified digital image, wherein the microscopically magnified digital image is obtained based on blood cells laid in a monolayer in a suspension; and the method includes: (6A): identifying multiple target cells in the microscopically magnified digital image; (6B): selecting a target image corresponding to each target cell in the microscopically magnified digital image; the target image including a target cell area and a blank area; (6C): based on a mean grayscale value of the target cell area in the target image and a mean grayscale value of the blank area in the target image, calculating a first absorbance of the target cell using a formula: the first absorbance of the target cell=log (the mean grayscale value of the blank area in the target image/the mean grayscale value of the target cell area in the target image); (8D): obtaining a first hemoglobin concentration correction coefficient; (8E): calculating a hemoglobin concentration of a single target cell using a formula: the hemoglobin concentration of a single target cell=the first absorbance×the first hemoglobin concentration correction coefficient;

In some embodiments, the step (8D) of obtaining the first hemoglobin concentration correction coefficient includes the following steps: (8DA1): taking an amount of blood cell sample to be analyzed; and obtaining a corpuscular hemoglobin, a red blood cell concentration per liter of the blood cell sample, and a mean corpuscular volume per liter of the blood cell sample using a hemoglobin analyzer; (8DA2): taking a same amount of blood cell sample to be analyzed as at step (8DA1), obtaining a cell suspension by pre-treating, and injecting the cell suspension into an imaging target area; making a monolayer of blood cells in the suspension, and obtaining a microscopically magnified digital image of the monolayer of blood cells in the suspension; (8DA3): selecting a target image corresponding to each target cell in the microscopically magnified digital image obtained at step (8DA2); the target image including a target cell area and a blank area; (8DA4): based on a mean grayscale value of each target cell area and a mean grayscale value of the blank area in each target image obtained at step (8DA3), calculating a first absorbance of each target cell using a formula: the first absorbance of each target cell=log (the mean grayscale value of the blank area/the mean grayscale value of each target cell area); (8DA5): calculating a mean value of the first absorbance based on the first absorbance of each target cell obtained at step (8DA4); and (8DA6): calculating a first hemoglobin concentration correction coefficient using a formula: the first hemoglobin concentration correction coefficient=the corpuscular hemoglobin per liter of blood cell sample÷the red blood cell concentration÷the mean corpuscular volume÷the mean value of the first absorbance.

A technical solution to solve the above problem is a hemoglobin analysis method based on a microscopically magnified digital image, the microscopically magnified digital image is obtained based on blood cells laid in a monolayer in a suspension, and the method includes the following steps: (9A): identifying a target cell area and a blank area in the microscopically magnified digital image; the target cell area including a target cell area corresponding to a single target cell and/or a target cell area corresponding to multiple cells overlapped; (9C): based on a mean grayscale value of the target cell area and a mean grayscale value of the blank area in the target image, calculating a first absorbance of the target cell using a formula: the first absorbance of the target cell=log (the mean grayscale value of the blank area/the mean grayscale value of the target cell area); (9I): obtaining a first hemoglobin absorption coefficient; (9J): calculating a corpuscular hemoglobin per unit area of the target cell area using a formula: the corpuscular hemoglobin per unit area of the target cell area=the first absorbance/the first hemoglobin absorption coefficient; (9K): obtaining a total target cell area and the number of target cells corresponding to the target cell area in the microscopically magnified digital image; (9L): calculating a corpuscular hemoglobin of each target cell using a formula: the corpuscular hemoglobin of each target cell=the total target cell area×the corpuscular hemoglobin per unit area of the target cell÷the number of target cells, or the corpuscular hemoglobin of each target cell=the total target cell area÷the number of target cells÷the first hemoglobin absorption coefficient×log (the mean grayscale value of the blank area/the mean grayscale value of the target cell area).

A technical solution to solve the above problem is an electronic device, including a memory, a processor, and a computer program stored in the memory and executable by the processor. The processor is configured to execute the computer program to realize The hemoglobin analysis method as described above.

A technical solution to solve the above problem is a readable storage medium, storing a computer program. The processor is configured to execute the computer program to realize the hemoglobin analysis method as described above.

### BRIEF DESCRIPTION OF THE DRAWINGS

FIG. 1 is a schematic diagram of the Beer-Lambert law.

FIG. 2 is a schematic block diagram of the principle of corpuscular hemoglobin measurement according to the related art.

FIG. 3 is a schematic diagram of a blood absorption spectrum, illustrating that blood exhibits distinct absorption peaks in several spectral regions, such as 420 nm, 540 nm, and 580 nm, and illustrating that the absorption peak near 418 nm is more prominent and obvious than the absorption peaks within 540 nm-580 nm range, which indicates that blood has a stronger absorption feature near 418 nm.

FIG. 4 is a schematic diagram of modules related to optical parts of a microscopic imaging system configured to obtain a microscopically magnified digital image.

FIG. 5 is a schematic diagram of an optical path of the microscopic imaging system based on FIG. 4.

FIG. 6 is a specific microscopically magnified digital image configured for hemoglobin analysis, illustrating that cells in the blood are in a monolayer state.

FIG. 7 is a schematic diagram of a microscopically magnified digital image in which multiple target cells are identified by AI algorithm and the identified target cells are highlighted with bounding boxes.

FIG. 8 is a schematic longitudinal sectional view of a cell in a solution under observation; b represents a length of the cell.

FIG. 9 and FIG. 10 are schematic diagrams of target images containing a target cell in FIG. 7; the target image is any selected image in FIG. 7; in FIG. 9 and FIG. 10, the circle represents a cell; the grid in FIG. 9 indicates an area; the outer frame in FIG. 10 represents the boundary of the target image, which includes the cell in the center and a blank area surrounding the cell.

FIG. 11 is a table comparing the results of the HiCN assay in the related art with the ANLV test method proposed in the present disclosure; multiple sample groups were tested for comparison.

FIG. 12 is a schematic diagram of a least squares linear regression analysis of the data presented in the table of FIG. 11; the scattered points represent HGB data from the ANLV test method, and the straight line represents HGB data from the HiCN test method; FIG. 8 illustrates a very strong correlation between the HGB data from the ANLV test method, and the HiCN assay method; the linear statistical chart calculates R²=0.9757, indicating a very strong correlation between the test data of the ANLV test method proposed by the present disclosure and the HiCN test method.

FIG. 13 is a histogram of the corpuscular hemoglobin (CH) of a single target cell of a cat blood sample.

FIG. 14 is a histogram of the corpuscular hemoglobin (CH) of a single target cell of a dog blood sample.

FIG. 15 is a histogram of the corpuscular hemoglobin (CH) of a single target cell of another dog blood sample; in FIG. 13 to FIG. 15, the horizontal axis represents the corpuscular hemoglobin (CH) of the target cell in units of pg, and the vertical axis represents the number of target cells in units of count.

FIG. 16 is a CV-CH scatter plot of a cat blood sample.

FIG. 17 is a CV-CH scatter plot of a dog blood sample

FIG. 18 is a CV-CH scatter plot of another dog blood sample.

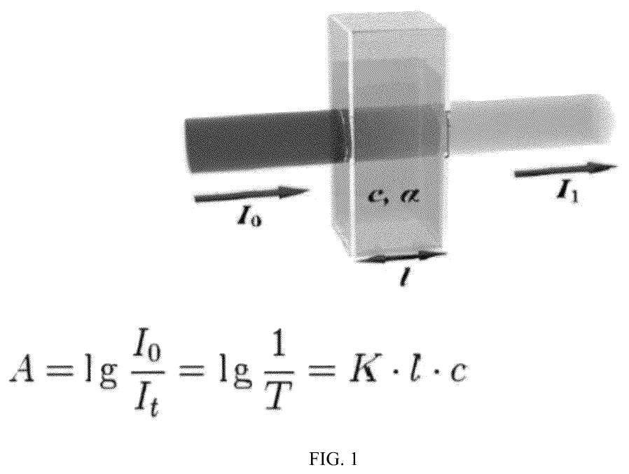

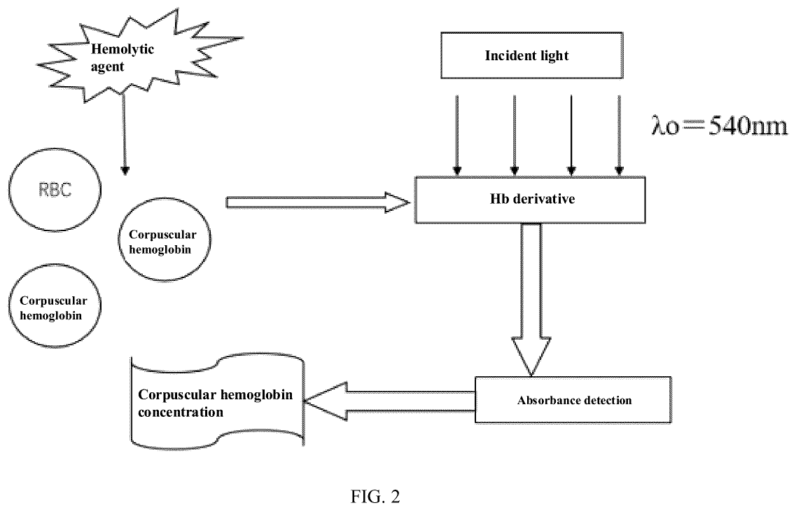

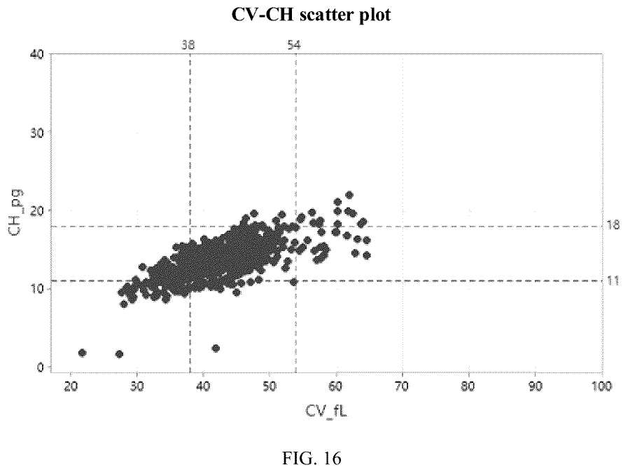

In FIG. 16 to FIG. 18, the vertical axis is the corpuscular hemoglobin (CH) of the target cells in units of pg, and the horizontal axis represents the volume of the target cells in units of fl.

### DETAILED DESCRIPTION OF THE EMBODIMENTS

The present disclosure is further described in detail below in conjunction with the accompanying drawings. It should be noted that the serial numbers in the method steps in the present disclosure are only for identification and distinction, and do not necessarily indicate a sequential relationship in time or space.

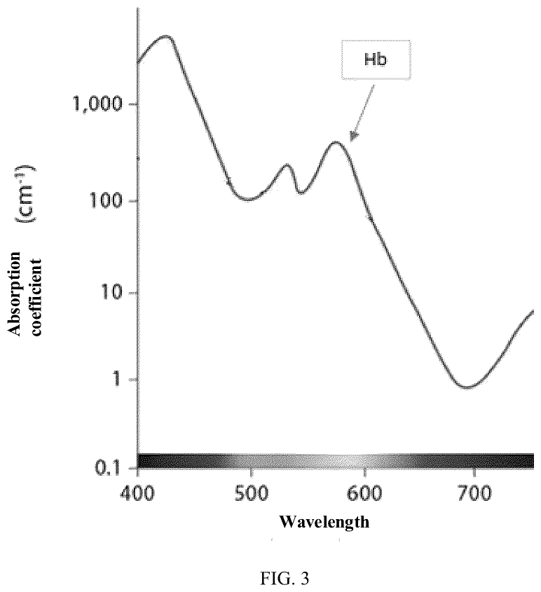

As artificial intelligence (AI) advances, leading it to be increasingly used in digital image processing. In the field of blood cell analysis, currently, no products that use AI algorithms are available to analyze cell parameters based on digital images of blood samples, especially those that use microscopically magnified digital images to analyze and measure hemoglobin concentration and content. The analysis and determination of hemoglobin concentration usually requires the use of the optical absorption characteristics of blood. As shown in FIG. 3, it is a schematic diagram of a blood absorption spectrum, illustrating that blood exhibits distinct absorption peaks near 418 nm and near 540 nm-580 nm, and that the absorption peak near 418 nm is more obvious and prominent than the absorption peaks between 540 nm-580 nm, which indicates that blood has a stronger absorption feature near 418 nm. In the related art, the absorption characteristics of Hb derivatives at specific wavelengths (530-550 nm) are usually utilized, while the absorption characteristics of blood itself are rarely utilized. The ferrous ions (Fe²⁺) in hemoglobin (except SHb) are oxidized into ferric ions (Fe³⁺) by potassium ferrocyanide contained in the hemolytic agent, converting hemoglobin into methemoglobin. Methemoglobin combines with cyanide ions (CN⁻) to form stable HiCN, i.e., a Hb derivative. The maximum absorption peak of HiCN is 540 nm. This combination determines that it is impossible to purely utilize the absorption characteristics of hemoglobin, especially the absorption characteristics near 418 nm. Due to the hemolysis process and the fact that the corpuscular hemoglobin is calculated based on the content of the hemoglobin derivatives rather than directly measuring the original corpuscular hemoglobin, measurement errors are introduced in the process.

If the cells in a blood sample can be spread out into a monolayer, each cell in the sample will have uniform non-scattering characteristics. Consequently, it is possible to perform content analysis based on the digital image obtained from the spread monolayer of cells. This approach eliminates the hemolysis process, simplifying the operational procedure; and allows for the selection of the wavelength band where the absorption characteristics of the blood cells are strongest for hemoglobin concentration analysis. As shown in FIG. 3, which depicts the absorption spectrum curve of blood cells, the strongest absorption peak of the blood cells is near 418 nm. Extracting absorption characteristics around this wavelength band will yield a better signal-to-noise ratio, making it easier to achieve higher measurement accuracy.

In the present disclosure, the basis for cell analysis based on a microscopically magnified digital image is that: diluting the blood cells to form a monolayer of cells while maintaining the original 3D morphology of the cells, photographing the monolayer of cells in a liquid matrix, and obtaining a bright-field microscopically magnified digital image; identifying the cell type based on the bright-field microscopically magnified digital image, analyzing and measuring the corpuscular hemoglobin.

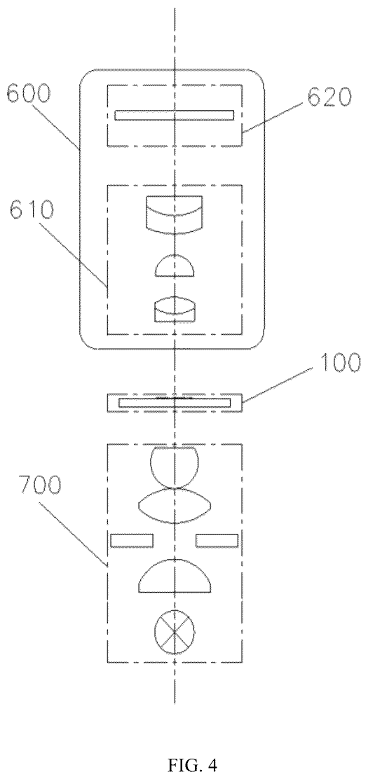

As shown in FIG. 4, it is a schematic diagram of modules related to optical parts of a microscopic imaging system configured to obtain a microscopically magnified digital image. In FIG. 4, reference numeral 600 represents a microscopic imaging module, reference numeral 620 represents a camera module, and reference numeral 610 represents a lens module; reference numeral 100 represents a target imaging area; reference numeral 700 represents an illumination lighting source module. The lens module 610 is disposed above the imaging target area to form a magnified microscopic image of the imaging target area; and the camera module 620 is configured to obtain digital image information of the magnified microscopic image. The lighting source module 700 can output at least two light beams, i.e., a first light beam and a second light beam, for illuminating the imaging target area according to the control instructions given by the main controller. The first light beam is a light beam with a first central wavelength of 418 nm; the second light beam is white light or a light beam with a second central wavelength. The white light is a mixed wide-spectrum light; the second central wavelength can also be a light beam with other central wavelengths, such as 540 nm, 580 nm, etc. The camera module includes either a black-and-white camera module or a color camera module. The illumination lighting source module is a wide-spectrum illumination light source; or the illumination lighting source module is a specific light source. The specific light source is a purple light source with a central wavelength including 418 nm; the central wavelength range of the specific light source is between 380 nm and 440 nm, or the central wavelength range of the specific light source is between 400 nm and 420 nm.

In some embodiments not shown in the accompanying drawings, the microscope imaging module further includes a narrow-band filter disposed in the optical path before light enters the camera module. The narrow-band filter can transmit light with a central wavelength range between 380 nm and 440 nm or between 400 nm and 420 nm.

Based on the above, the microscopically magnified digital image is obtained under the illumination of a wide-spectrum illumination light source; or the microscopically magnified digital image is obtained under the illumination of a specific light source. The specific light source is a purple light source with a central wavelength including 418 nm; the central wavelength range of the specific light source is between 380 nm and 440 nm or between 400 nm and 420 nm. The microscopically magnified digital image is an R/G/B three-channel microscopically magnified digital image containing at least three-color components, and the R/G/B three channels are respectively a red channel, a green channel and a blue channel.

A specific light source with a central wavelength range of 380 nm to 440 nm is a purple light source with a central wavelength including 418 nm. The incident light source enhances the light intensity near the hemoglobin absorption peak band, further highlighting the change in light absorption near the absorption peak and improving the signal-to-noise ratio, thereby obtaining a more accurate calculation result.

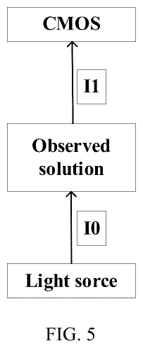

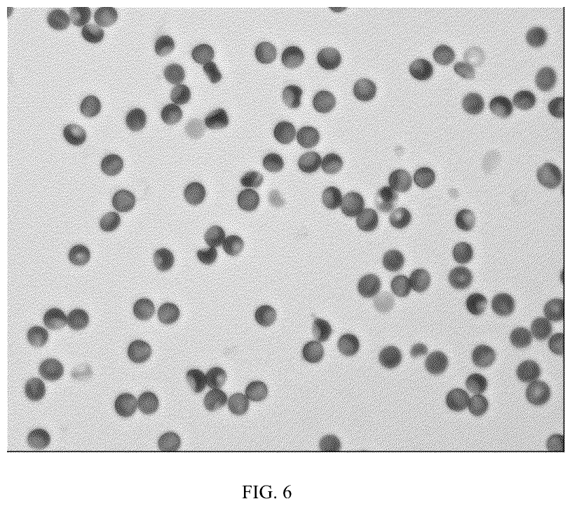

FIG. 5 illustrates an optical path of the microscopic imaging system based on FIG. 4; the initial light intensity I₀ from the light source passes through the observed solution, with the absorbed portion of the light intensity being the absorbed light intensity Id. The transmitted light intensity I1 enters the CMOS imaging unit of the camera module to obtain a microscopically magnified digital image. The observed solution is a suspension containing a monolayer of cells, which is a diluted blood sample. The blood diluent also includes a staining solution with staining functionality.

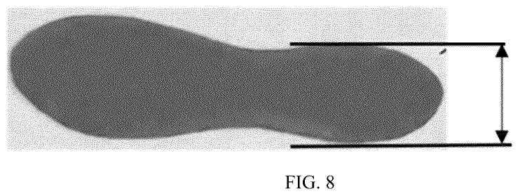

FIG. 8 is a schematic longitudinal sectional view of a cell in a solution under observation. In FIG. 8, the cell is in the middle, which is surrounded by a suspension. A thickness of the cell is b.

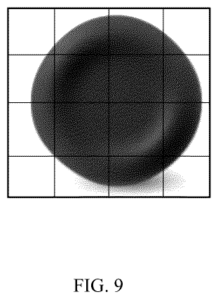

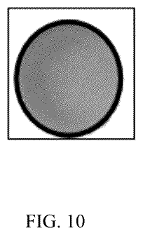

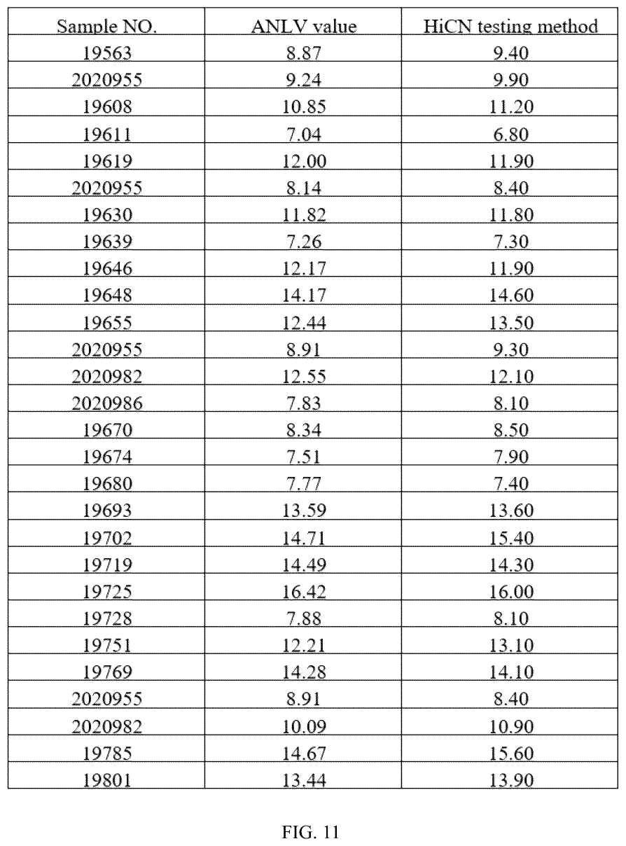

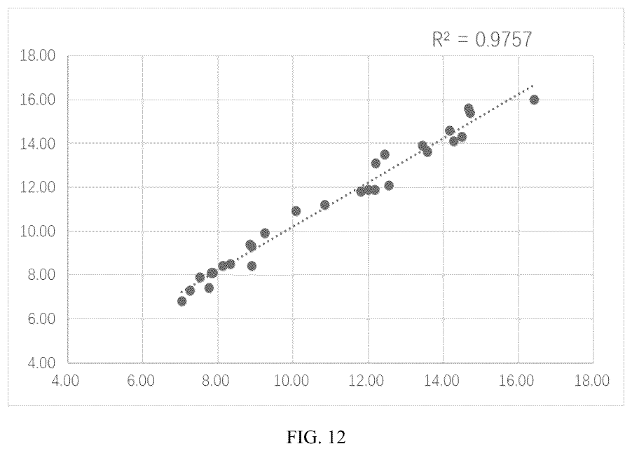

FIG. 9 is a schematic view of a target image containing a target cell; I₀ is an incident light intensity, which is represented by a grayscale value of a blank area in the algorithm of the present disclosure; It is a transmitted light intensity, which is represented by a grayscale value of a cell area in the algorithm of the present disclosure.

The microscopically magnified digital image described in the present disclosure may be a grayscale image; each pixel in the image may have a grayscale value ranging from 0 (black) to 255 (white). 0-255 represents different grayscale levels. The microscopically magnified digital image described in the present disclosure may also be a color image, which is composed of three grayscale images corresponding to different color channels, with one grayscale image corresponding to the red channel, one grayscale image corresponding to the green channel, and another grayscale image corresponding to the blue channel.

In an embodiment, a hemoglobin analysis method based on a microscopically magnified digital image is configured to calculate the corpuscular hemoglobin concentration, the microscopically magnified digital image is obtained based on a monolayer of blood cells spread in a suspension, i.e., the blood cells in the microscopically magnified digital image are roughly spread out in a monolayer. The suspension can be regular physiological saline or a specific diluent containing or not containing a dye. The suspension, in principle, only requires that the absorption spectra of other substances do not overlap with the that of the target cells.

The microscopically magnified digital image can also be obtained under the illumination of a wide-spectrum illumination source. The microscopically magnified digital image contains multi-color component information in the R/G/B three channels. Whether under illumination by a specific light source with a defined central wavelength range or under wide-spectrum illumination, it is sufficient if the central wavelength range includes 418 nm or the absorption peaks of other blood cells.

In the present disclosure, the basic data required for corpuscular hemoglobin measurement is converted from the light intensity obtained by a specific optical detector to the grayscale value in the digital image, greatly simplifying the hardware structure of the entire device. In other words, the corpuscular hemoglobin is analyzed and measured with extremely low hardware costs. Moreover, this method is based on bright-field imaging, which is very intuitive and offers better accuracy. It neither involves the complex design of a spectrophotometer nor requires hemolytic agents for the release and binding of hemoglobin. The entire technical solution is extremely simple, with high efficiency from development to usage and maintenance, and the cost is very low.

Since the imaging is directly done on blood cells without the hemolysis process, it leverages the blood's natural characteristics at the 418 nm absorption peak. This allows for the maximum grayscale variation in digital image processing, enabling accurate grayscale value calculations with a relatively good signal-to-noise ratio.

In an embodiment, the hemoglobin analysis method based on a microscopically magnified digital image includes step 6A: identify multiple target cells in a microscopically magnified digital image. The hemoglobin analysis is performed based on the identified target cells; the identified target cells include red blood cells and reticulocytes. In some embodiments, the identified target cells include red blood cells. In some embodiments, the identified target cells include and reticulocytes. In some embodiments, the identified target cells include red blood cells and reticulocytes. The method for identifying multiple target cells in a microscopically magnified digital image can be a traditional image processing method or AI algorithms. There are relatively mature recognition and counting algorithms based on AI algorithms for identifying and counting cell types in microscopically magnified digital images. Any algorithm in the related art can be used and will not be described in detail here. To identify multiple target cells in microscopically magnified digital images, traditional image recognition methods or AI algorithms can be used.

In an embodiment, the hemoglobin analysis method based on a microscopically magnified digital image includes the following steps. Step 6B: select a target image corresponding to each target cell in the microscopically magnified digital image; the target image includes a target cell area and a blank area. Step 6C: based on a mean grayscale value (Gc) of the target cell area in the target image and a mean grayscale value (Gb) of the blank area in the target image, calculating a first absorbance (α1) of the target cell using a formula: α1=log (Gb/Gc). Here, Gc is equivalent to the intensity of transmitted light; Gb is equivalent to the intensity of incident light. At step 6B, each target cell in the target image is an independent cell. The cell in the target image corresponding to each target cell is an independent cell. Cells which are identified with overlapping regions will not be used in the subsequent calculation.

In some embodiments, the hemoglobin analysis method based on a microscopically magnified digital image includes the following steps. Step 6I: obtain a first hemoglobin absorption coefficient KHGB. Step 6J: calculate a corpuscular hemoglobin be per unit area of the target cell using a formula: bc=α1/KHGB; here, al represents the first absorbance, KHGB represents first hemoglobin absorption coefficient. Step 6K: obtain each target cell area (STC) in the microscopically magnified digital image. Step 6L: calculate a corpuscular hemoglobin (CH) of each target cell using a formula: CH=STC×bc, i.e.,

$$CH = \frac{STC}{{K}_{HGB}} lg \frac{Gb}{Gc} ;$$

here, STC represents each target cell area, KHGB represents the first hemoglobin absorption coefficient, Gb represents the mean grayscale value of the blank area; Gc represents the mean grayscale value of the target cell area.

In the above calculations, skillfully, the corpuscular hemoglobin be per unit area of the target cell is calculated first and used as a calculation unit in the subsequent calculations of the corpuscular hemoglobin (CH) of the target cell. As a calculation unit, it is directly multiplied with the target cell area (STC) to obtain the corpuscular hemoglobin per unit volume. The measurement and calculation process of the length on the absorption light path of a single cell is avoided, reducing the errors introduced thereby.

In some embodiments, the hemoglobin analysis method based on a microscopically magnified digital image further includes the following steps. Step 6JA: obtain a first hemoglobin absorption coefficient K_{HGB}. Step 6JA includes the following steps. Step 6IA1: take an amount of blood cell sample to be analyzed and obtain the corpuscular hemoglobin (HGB) and the red blood cell concentration (RBC) per liter of the blood cell sample. Step 6IA2: take a same amount of cell sample to be analyzed as step 6IA1 by a hemoglobin analyzer, obtain a cell suspension by pre-treating, and inject the cell suspension into the imaging target area; make a monolayer of blood cells in the suspension, and obtain a microscopically magnified digital image of the monolayer of blood cells in the suspension. Step 6IA3: select a target image corresponding to each target cell in the microscopically magnified digital image obtained at step 6IA2; the target image includes a target cell area and a blank area. Step 6IA4: calculate a first absorbance al of each target cell using the mean grayscale value Gc of each target cell area and the mean grayscale value Gb of the blank area in each target image obtained at step 6IA3 using a formula: α1=log (Gb/Gc). Step 6IA5: obtain the mean of the first absorbance al of each target cell obtained at step 6IA4. Step 6IA6: calculate the mean area of all target cells (SVTC) based on each target cell area (STC). Step 6IA7: calculate a first hemoglobin absorption coefficient (KHGB) using a formula: K_{HGB}=α1÷bc, here, α1 represents the mean of the first absorbance, be represents the corpuscular hemoglobin per unit area; K_{HGB}=α1÷((HGB)÷RBC÷SVTC)=α1×RBC×SVTC+(HGB), where al represents the mean value of the first absorbance, HGB represents the corpuscular hemoglobin per liter of blood cell sample, RBC represents red blood cell concentration, SVTC represents mean area of target cells.

The first hemoglobin absorption coefficient K_{HGB} is a constant value corresponding to the target sample to be tested, or a constant value corresponding to the target sample to be tested obtained by looking up a data table.

In some embodiments, the hemoglobin analysis method based on a microscopically magnified digital image includes the following steps. Step 7I: obtain a first corpuscular hemoglobin correction coefficient (CHGB1). Step 7K: obtain each target cell area (STC) in the microscopically magnified digital image. Step 7J: calculate a target cell corpuscular hemoglobin (CH) using a formula: CH=α1×STC×CHGB1, here, al represents the first absorbance, STC represents each target cell area, CHGB1 represents the first corpuscular hemoglobin correction coefficient.

In some embodiments, the hemoglobin analysis method based on a microscopically magnified digital image further includes the following steps. Step 7JA: obtain a first corpuscular hemoglobin correction coefficient (CHGB1). Step 7JA includes the following steps. Step 7IA1: take an amount of blood cell sample to be analyzed, obtain a corpuscular hemoglobin (HGB) and a red blood cell concentration (RBC) per liter of the blood cell sample using a hemoglobin analyzer. Step 7IA2: take a same amount of cell sample to be analyzed as at step 7IA1, obtain a cell suspension by pre-treating, inject the cell suspension into the imaging target area; make a monolayer of blood cells in the suspension, and obtain a microscopically magnified digital image of the monolayer of blood cells in the suspension. Step 7IA3: select a target image corresponding to each target cell in the microscopically magnified digital image obtained at step 7IA2, the target image includes a target cell area and a blank area. Step 7IA4: based on a mean grayscale value (Gc) of each target cell area and a mean grayscale value (Gb) of the blank area in each target image in the target image obtained at step 6IA3, calculate a first absorbance (α1) using a formula: α1=log (Gb/Gc). Step 7IA5: obtain a mean value of the first absorbance (α1) based on the first absorbance (α1) of each target cell obtained at step (7IA4). Step 7IA6: obtain each target cell area (STC) and calculate a mean area of the target cells (SVTC). Step 7IA7: calculating a first corpuscular hemoglobin correction coefficient (CHGB1) using a formula: CHGB1=HGB÷(RBC)÷α1÷SVTC, here, CHGB1 represents the first corpuscular hemoglobin correction coefficient, HGB represents the corpuscular hemoglobin per liter of blood cell sample, RBC represents the red blood cell concentration, al represents the mean value of the first absorbance, SVTC represents mean area of the target cells.

The first corpuscular hemoglobin correction coefficient (CHGB1) is a constant value corresponding to the target sample to be tested, or a constant value corresponding to the target sample to be tested obtained by looking up a data table.

In some other embodiments, the hemoglobin analysis method based on a microscopically magnified digital image includes the following steps. Step 8D: obtain a first hemoglobin concentration correction coefficient (CHC1). Step 8E: calculate a hemoglobin concentration of a single target cell (CHGBs) using a formula: CHGBs=α1×CHC1, where α1 represents the first absorbance, CHC1 represents the first hemoglobin concentration correction coefficient. The first hemoglobin concentration correction coefficient (CHC1) is a known parameter, equivalent to the product Kb in the Beer-Lambert law, where K represents the molar absorption coefficient, and b represents the absorption layer thickness. For individual cells, the thickness varies; however, statistically, the mean thickness of target cells is approximately constant.

In some embodiments, the hemoglobin analysis method based on a microscopically magnified digital image further includes the following steps. Step 8DA: obtain a first hemoglobin concentration correction coefficient (CHC1). Step 8DA includes the following steps. Step 8DA1: take an amount of blood cell sample to be analyzed, and obtain a corpuscular hemoglobin (HGB), a red blood cell concentration (RBC) and a mean corpuscular volume (MCV) per liter of the blood cell sample using a hemoglobin analyzer. Step 8DA2: take a same amount of cell sample to be analyzed as at step 8DA1, obtain a cell suspension by pre-treating, inject the cell suspension into an imaging target area, make a monolayer of blood cells in the suspension, and obtain a microscopically magnified digital image of the monolayer of blood cells in the suspension. Step 8DA3: select a target image corresponding to each target cell in the microscopically magnified digital image obtained at step 8DA2, and the target image includes a target cell area and a blank area. Step 8DA4: based on a mean grayscale value Gc of each target cell area and a mean grayscale value Gb of the blank area in each target image in the target image obtained at step (8DA3), calculate a first absorbance α1 of each target cell using a formula: α1=log (Gb/Gc), here, Gb represents mean grayscale value of the blank area, Gc represents mean grayscale value of the target cell area. Step 8DA5: calculating a mean value of the first absorbance α1 based on the first absorbance α1 of each target cell obtained at step 8DA4. Step 8DA6: calculate a first hemoglobin concentration correction coefficient CHC1 using a formula: CHC1=HGB+RBC+MCV+mean value of α1, CHC1 represents first hemoglobin concentration correction coefficient, HGB represents corpuscular hemoglobin per liter of blood cell sample, MCV represents mean corpuscular volume. The first hemoglobin concentration correction coefficient (CHC1) is a constant value corresponding to the target sample to be tested, or a constant value corresponding to the target sample to be tested obtained by looking up a data table.

In some embodiments, the hemoglobin analysis method based on a microscopically magnified digital image includes the following steps. Step 6F: obtain hemoglobin concentration (CHGBs) of all target cells in the microscopically magnified digital image, and calculate a mean corpuscular hemoglobin concentration (MCHC) using a formula: MCHC=Σ(CHGBs)÷NTC, here, MCHC represents a mean corpuscular hemoglobin concentration, and NTC represents the number of all target cells. Step 6G: identify the target cells in the microscopically magnified digital image using AI algorithm, obtain a single target cell area (STC) in the microscopically magnified digital image, and obtain a known mean cell height b. Step 6H: calculate a corpuscular hemoglobin (CH) of a single target red blood cell using a formula: CH=STC×CHGBs×b, where STC represents single target cell area, CHGBs represents single target cell hemoglobin concentration, b represents mean cell height. Σ(CHGBs) represents the sum of the hemoglobin concentration of all target cells (CHGBs).

In some embodiments, the hemoglobin analysis method based on a microscopically magnified digital image includes the following step. Step 6M2: output a histogram of the corpuscular hemoglobin (CH) of each target cell according to the corpuscular hemoglobin (CH) of each target cell; the histogram is configured to count the hemoglobin distribution patterns of different target cells.

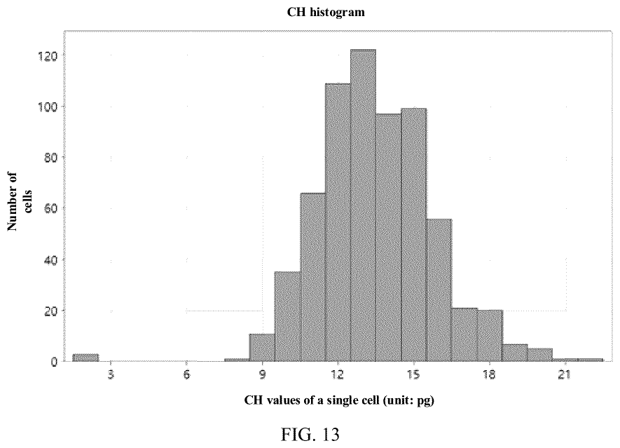

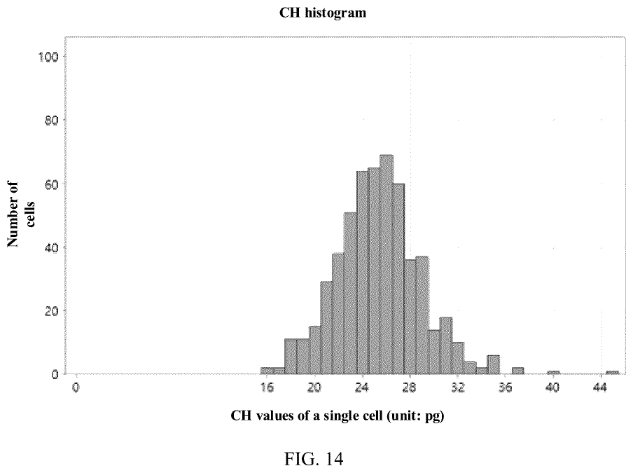

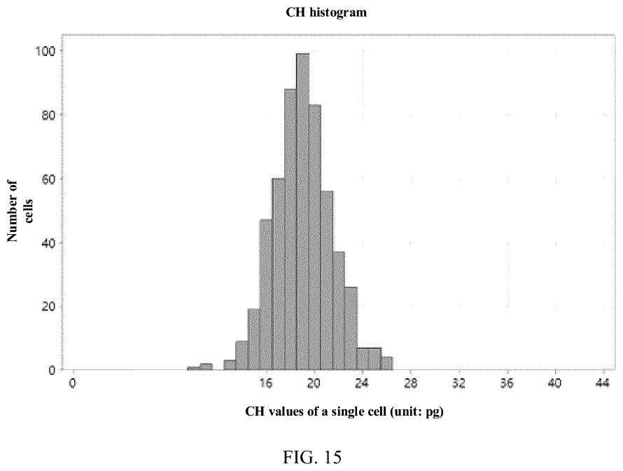

The sample involved in FIG. 13 is a normal healthy cat blood sample, and the corpuscular hemoglobin (HGB) is within the normal range. The sample involved in FIG. 14 is a normal healthy dog blood sample. The sample involved in FIG. 15 is another dog blood sample. FIG. 15 illustrates that the distribution of individual hyperchromic red blood cells is shifted to the left, and the MCH value is lower than the reference value range (22 pg-27 pg), indicating the possibility of anemia.

Although not shown in the attached drawings, in actual applications, the center of the CH distribution of the corpuscular hemoglobin of a single target cell shifting left or right often indicates some abnormalities. This abnormal shift information is usually one of the manifestation characteristics of clinical pathology. An accurate CH histogram of the corpuscular hemoglobin of a single target cell offers an easier distribution of the corpuscular hemoglobin (CH) of the target cell, and the central content position of the corpuscular hemoglobin (CH) of the target cell can also reflect the pathological characteristics of the sample.

With the information of the corpuscular hemoglobin (CH) of a single target cell, accurate statistical analysis can be performed. This allows the output of a histogram of the corpuscular hemoglobin (CH) of a single target cell, as shown in FIG. 13 to FIG. 15, providing more detailed data and statistical information reference for further clinical hemoglobin analysis and research in clinical settings.

In some embodiments, the hemoglobin analysis method based on a microscopically magnified digital image includes the followings steps. Step 6M3: obtain a volume of each target cell and output a CH-CV scatter plot according to the volume of each target cell and the corpuscular hemoglobin (CH) of each target cell. The CH-CV scatter plot is configured to statistically analyze a hemoglobin distribution pattern of target cells with different volumes. Step 6M4: display at least one CH indicator line and at least one CV indicator line on the CH-CV scatter plot. The CH-CV scatter plot provides statistical reference information for clinical anemia research. Especially when combined with the CH indicator line and the CV indicator line, the normal range of CV-CH can be indicated by clear lines, which is very intuitive for clinicians.

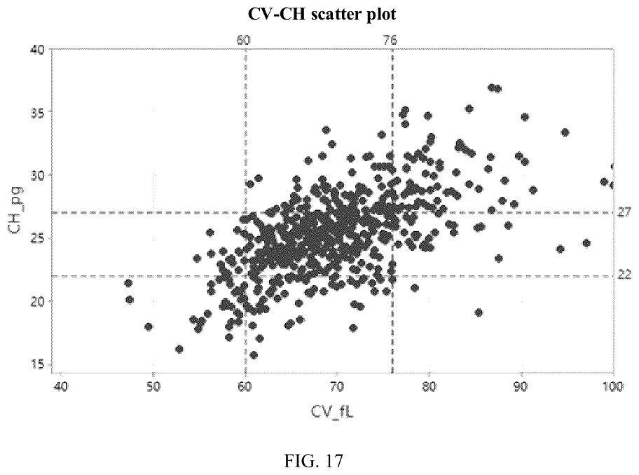

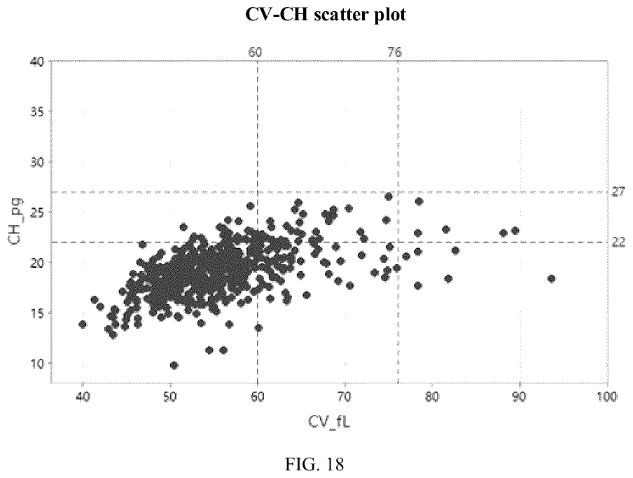

FIG. 16 shows a schematic diagram of the CV-CH scatter plot of a healthy cat blood sample, illustrating the CH reference range of a normal cat being 38 to 54 pg, corresponding to two CV indicator lines; the CV reference range being 11 to 18 fL, corresponding to two CH indicator lines. The reference range displayed on the CH-CV scatter plot clearly shows the distribution trend, which is very intuitive for clinical purposes and convenient for doctors to refer to. FIG. 17 shows a schematic diagram of a CV-CH scatter plot of a healthy dog blood sample; and shows the CH reference range of a normal dog is 22 to 27 μg and the CV reference range is 60 to 76 fL. The reference range displayed on the CH-CV scatter plot clearly shows the distribution trend, which is very intuitive for clinical purposes and convenient for doctors to refer to. FIG. 18 shows a schematic diagram of a CV-CH scatter plot of another dog blood sample, mainly concentrating on the lower left corner, and reflecting smaller CH and CV values. The clinical manifestations are microcytic anemia or microcytic hypochromic anemia. Common diseases may include chronic infection, poisoning, inflammation, liver disease, uremia, malignant tumors, rheumatic diseases; chronic inflammation, uremia; iron deficiency anemia, chronic hemolysis, globin production disorder anemia, sideroblastic anemia, etc. In some embodiments, the hemoglobin analysis method based on a microscopically magnified digital image includes the following steps. Step 6M: obtain a mean corpuscular hemoglobin (MCH) of the target cells based on summing up a total of the corpuscular hemoglobin (CH) of each target cell. Step 6N: obtain a mean corpuscular volume (MCV). Step 6P: calculate a mean corpuscular hemoglobin concentration (MCHC) using a formula: MCHC=MCH÷MCV, here, MCH represents mean corpuscular hemoglobin of target cells, MCV represents mean corpuscular volume.

In some embodiments, the hemoglobin analysis method based on a microscopically magnified digital image includes the following steps. Step 6C: based on the mean grayscale value of the blue channel of the target image and the mean grayscale value of the blue channel of the blank area of the target image, calculate a first absorbance α1 of a target cell using a formula: α1=log (the mean grayscale value of the blue channel of the blank area/the mean grayscale value of the blue channel of the target cell area). Step 6IA4: based on the mean grayscale value of the blue channel of each target cell area and the mean grayscale value of the blue channel of the blank area in each target image in the target image obtained at step 6IA3, calculate the first absorbance α1 of each target cell using a formula: α1=log (the mean grayscale value of the blue channel of the blank area/the mean grayscale value of the blue channel of the target cell area). Step 7IA4: based on the mean grayscale value Gc of the blue channel of each target cell area and the mean grayscale value Gb of the blue channel of the blank area in each target image in the target image obtained at step 7IA3, calculate the first absorbance α1 of each target cell using a formula: α1=log (the mean grayscale value of the blue channel of the blank area Gb/the mean grayscale value of the blue channel of the target cell area Gc). Step 8DA4: based on the mean grayscale value Gc of the blue channel of each target cell area and the mean grayscale value Gb of the blue channel of blank area in each target image in the target image obtained at step 8DA3, calculate the first absorbance α1 of each target cell using a formula: α1=log (the mean grayscale value of the blue channel of blank area Gb/the mean grayscale value of the blue channel of target cell area Gc). Since the characteristic absorption peaks of hemoglobin are 418 nm and between 530-560 nm, for microscopically magnified digital images, the characteristic absorption peaks presented in the blue channel will be more obvious than those in the red channel. Therefore, the blue channel and the grayscale value of any channel are configured to calculate the corresponding parameters. The blue channel can highlight the spectral characteristic information near the 418 nm absorption peak. As the image signal-to-noise ratio of the blue channel is relatively high, only the blue channel is configured for calculation, which improves the calculation efficiency.

In some embodiments, the hemoglobin analysis method based on a microscopically magnified digital image includes the following steps. Step 6C: based on the mean grayscale value of any channel of the target cell area in the target image and the mean grayscale value of any channel of the blank area in the target image, calculate a first absorbance α1 of the target image using a formula: α1=log (the mean grayscale value of any channel of the blank area/the mean grayscale value of any channel of the target cell area). Step 6IA4, based on the mean grayscale value of any channel of each target cell area and the mean grayscale value of any channel of the blank area in each target image in the target image obtained at step 6IA3, calculate the first absorbance α1 of each target cell using a formula: α1=log (the mean grayscale value of any channel of the blank area/the mean grayscale value of any channel of the target cell area). Step 7IA4: based on the mean grayscale value Gc of any channel of the target cell area and the mean grayscale value Gb of any channel of the blank area in each target image in the target image obtained at step 7IA3, calculate the first absorbance α1 of each target cell using a formula: α1=log (the mean grayscale value Gb of any channel of the blank area/the mean grayscale value Gc of any channel of the target cell area). Step 8DA4, based on the mean grayscale value of any channel of the target cell area and the mean grayscale value of any channel of the blank area in each target image in the target image obtained at step 8DA3, calculate the first absorbance α1 of each target cell using a formula: α1=log (the mean grayscale value of any channel of the blank area/the mean grayscale value of any channel of the target cell area). Any channel includes a red channel, a green channel and a blue channel. The information of any channel includes both the spectral characteristic information near the absorption peak at 418 nm and the spectral characteristic information between 530-560 nm, which can integrate the absorption of blood cells at each absorption peak for subsequent calculations. A single channel for calculation can reduce the amount of computation; in addition, the characteristics of white light or other wide-spectrum light sources are considered to ensure that the corresponding absorption characteristic information can be extracted.

In some embodiments, a hemoglobin analysis method based on a microscopically magnified digital image includes the following steps. The microscopically magnified digital image is obtained based on a monolayer of blood cells laid in a suspension. Step 9A: identify a target cell area and a blank area in the microscopically magnified digital image; the target cell area includes a target cell area A corresponding to a single target cell and/or a target cell area B corresponding to multiple cells overlapped; or the target cell area only selects a target cell area A corresponding to a single target cell. Step 9C: based on the mean grayscale value of the target cell area Gc and the mean grayscale value Gb of the blank area in the target image, calculate a first absorbance α1 using a formula: α1=log (the mean grayscale value Gb of the blank area/the mean grayscale value Gc of the target cell area). Step 9I: obtain a first hemoglobin absorption coefficient K_{HG}B. Step 9J: calculate a corpuscular hemoglobin per unit area of the target cell area be using a formula: bc=first absorbance α1/first hemoglobin absorption coefficient K_{HGB}. Step 9K: obtain a total target cell area (ASTC) and the number of target cells (NC) corresponding to the target cell area in the microscopically magnified digital image. Step 9L: calculate a corpuscular hemoglobin of each target cell CH using a formula: CH=ASTC×bc÷NC, or CH=ASTC÷NC÷K_{HGB}×log (Gb/Gc), ASTC represents the total target cell area, be represents the corpuscular hemoglobin per unit area of target cells, NC represents the number of target cells.

The target cell area A corresponding to a single target cell refers to a situation where the target cell area A is independently displayed by a single target cell, and there are as many target cell areas A as there are independent cells. The target cell area B with multiple cells overlapped refers to a whole target cell area B formed by two or more cells adhering together; there are as many target cell areas B as there are cell overlapping areas.

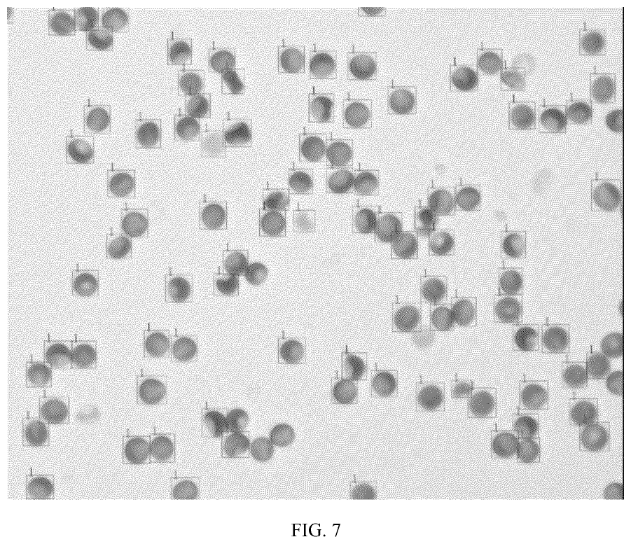

As shown in FIG. 7, most of the cells are independently dispersed, and such an independent single target cell corresponds to target cell area A; while some cells are overlapped, and such overlapped multi-cells correspond to target cell area B. For calculation of corpuscular hemoglobin, whether using the target cell area A alone, or combined with the target cell area B, or using target cell area B alone, the corpuscular hemoglobin (CH) of each target cell can be measured.

In the present disclosure, a hemoglobin analysis method based on a microscopically magnified digital image includes: identifying multiple target cells in the microscopically magnified digital image; selecting a target image corresponding to each target cell; the target image includes a target cell area and a blank area; calculating a first absorbance α1 of the target cell using a formula: log (the mean grayscale value Gb of the blank area/the mean grayscale value Gc of the target cell area); obtaining each target cell area (STC) in the microscopically magnified digital image, and calculating the corpuscular hemoglobin of each target cell as:

$$\frac{STC}{{K}_{HGB}} lg \frac{Gb}{Gc} .$$

The corpuscular hemoglobin of the target cell CH=α1×STC×CHGB1, here, al represents the first absorbance, STC represents the target cell area, CHGB1 represents the first corpuscular hemoglobin correction coefficient. CHGBs=α1×CHC1, CHGBs represents the target cell hemoglobin concentration, al represents the first absorbance, CHC1 represents the first hemoglobin concentration correction coefficient. By combining the Beer-Lambert law with digitally magnified microscopic imaging, the entire measurement system is simplified. This approach makes the optical and fluidic paths maintenance-free and streamlines the operation and control processes, significantly enhancing the efficiency of hemoglobin test.

Compared with the related art, one of the beneficial technical effects of the present disclosure is that, based on a microscopically magnified digital image obtained by a monolayer of blood cells spread out in a suspension, the Beer-Lambert law and the microscopically magnified digital image are combined and applied to obtain the corpuscular hemoglobin of a single target cell, allowing the perspective of clinical observation of the corpuscular hemoglobin to be extended from the corpuscular hemoglobin of the entire sample to the corpuscular hemoglobin at the level of a single cell. Moreover, the cell morphology in the cell suspension is intact, enabling a direct measurement of hemoglobin of a single intact cell, which has high accuracy and does not require hemolysis.

Compared with the related art, the second beneficial technical effect of the present disclosure is that it can obtain the corpuscular hemoglobin of a single target cell, resulting in an accurate basis for the statistical analysis of the single corpuscular hemoglobin, and also enabling the statistical analysis of the corpuscular hemoglobin of a single cell to be carried out, thereby delving into a deeper level of valuable information for clinical use. In the classification of various anemias, the statistical analysis of the corpuscular hemoglobin of a single cell shows clinical significance.

Compared with the related art, the third beneficial technical effect of the present disclosure is that, in the cell suspension, the cell morphology remains intact, contributing to a more accurate the cell volume measurement. In addition, the combination of the single cell volume and the single cell corpuscular hemoglobin realizes the statistical analysis of the single corpuscular hemoglobin and the single hemoglobin volume, delving into deeper multi-dimensional valuable information for clinical use. Especially in the classification of various anemias, the statistical analysis of the corpuscular hemoglobin of a single cell combined with the volume of a single hemoglobin has extremely significant clinical value.

Compared with the related art, the fourth beneficial technical effect of the present disclosure is that AI algorithm can identify single target cells in microscopically magnified digital images, and each target cell in the target image is an independent single cell. Therefore, the target cell area (STC), the first absorbance α1 of the target cell, and the target cell corpuscular hemoglobin (CH) can all be calculated at the level of a single cell. Additionally, the calculation is more precise; and as the number of examples in the AI algorithm increases and becomes richer, the accuracy of the calculation will also increase accordingly.

Compared with the related art, the fifth beneficial technical effect of the present disclosure is that the first hemoglobin absorption coefficient K_{HGB}, the first corpuscular hemoglobin correction coefficient (CHGB1) and the first hemoglobin concentration correction coefficient (CHC1) can obtain the corresponding parameters of different types of target cells by looking up a table, which simplifies the calculation process and improves the overall calculation efficiency.

Compared with the related art, the sixth beneficial technical effect of the present disclosure is that it can obtain the first hemoglobin absorption coefficient K_{HGB}, the first corpuscular hemoglobin correction coefficient (CHGB1) and the first hemoglobin concentration correction coefficient (CHC1) by introducing a hemoglobin analyzer with equivalent accuracy or higher accuracy, thereby ensuring the consistency and accuracy of the coefficients, which is more suitable for corpuscular hemoglobin measurement. These coefficients can also be obtained according to the sample type by introducing benchmark instruments for comparative references, further improving the compatibility and scalability of the system and making it applicable to test the corpuscular hemoglobin of a variety of samples.

Compared with the related art, the seventh beneficial technical effect of the present disclosure is that the central wavelength range of the specific light source is between 380 nm and 440 nm, or the central wavelength setting of the filter fully utilizes the characteristics of the light source and the filter, naturally enhances the original signal at 418 nm, which is the strongest absorption peak of blood cells, improves the image quality, especially the signal-to-noise ratio at the strongest absorption peak, and can improve the accuracy of measurement and calculation.

Compared with the related art, the eighth beneficial technical effect of the present disclosure is that the microscopically magnified digital image obtained under white light or other wide-spectrum light sources uses the blue channel or any channel of the microscopically magnified digital image, which utilizes the digital characteristics of the image, equivalent to filtering out the corresponding light signals of other channels, and disguisedly enhancing the relative signal amount at 418 nm, i.e., the strongest absorption peak of blood cells. Therefore, the signal-to-noise ratio at the strongest absorption peak is improved, further enhancing the measurement and calculation accuracy.

Compared with the related art, the ninth beneficial technical effect of the present disclosure is that the target cell mean corpuscular hemoglobin (MCH) is calculated by the corpuscular hemoglobin (CH) of each target cell, and the mean corpuscular hemoglobin concentration (MCHC) is calculated by the hemoglobin concentration (CHGBs) of each target cell. Both are direct calculation and measurement processes, which are closer to the actual situation and avoid the process errors introduced by conversion when measuring through derivatives.

The above descriptions are merely embodiments of the present disclosure and are not intended to limit the scope of the present disclosure. Any equivalent structure or equivalent process transformation made using the contents of the specification and drawings, or directly or indirectly applied in other related technical fields, are also included in the scope of protection of the present disclosure.

What is claimed is:

1. A hemoglobin analysis method based on a microscopically magnified digital image, wherein the microscopically magnified digital image is obtained based on blood cells laid in a monolayer in a suspension; and the method comprises the following steps:
(6A): identifying multiple target cells in the microscopically magnified digital image;
(6B): selecting a target image corresponding to each target cell in the microscopically magnified digital image; wherein the target image comprises a target cell area and a blank area;
(6C): based on a mean grayscale value of the target cell area in the target image and a mean grayscale value of the blank area in the target image, calculating a first absorbance of the target cell using a formula: the first absorbance of the target cell=log (the mean grayscale value of the blank area in the target image/the mean grayscale value of the target cell area in the target image);
(6I): obtaining a first hemoglobin absorption coefficient;
(6J): calculating a corpuscular hemoglobin per unit area of the target cell using a formula: the corpuscular hemoglobin per unit area of the target cell=the first absorbance of the target cell/the first hemoglobin absorption coefficient;
(6K): obtaining each target cell area in the microscopically magnified digital image; and
(6L): calculating a corpuscular hemoglobin of each target cell using a formula: the corpuscular hemoglobin of each target cell=each target cell area×the corpuscular hemoglobin per unit area of the target cell, or the corpuscular hemoglobin of each target cell=each target cell area/the first hemoglobin absorption coefficient×log (the mean grayscale value of the blank area in the target image/the mean grayscale value of the target cell area in the target image);
wherein the step (6I) of obtaining the first hemoglobin absorption coefficient comprises the following steps:
(6IA1): taking an amount of blood cell sample to be analyzed, and obtaining a corpuscular hemoglobin and a red blood cell concentration per liter of the blood cell sample using a hemoglobin analyzer;
(6IA2): taking a same amount of the blood cell sample to be analyzed as at step (6IA1), obtaining a cell suspension by pre-treating, injecting the cell suspension into an imaging target area; making a monolayer of blood cells in the suspension, and obtaining a microscopically magnified digital image of the monolayer of blood cells in the suspension;
(6IA3): selecting a target image corresponding to each target cell in the microscopically magnified digital image obtained at step (6IA2), wherein the corresponding target image comprises a target cell area and a blank area;
(6IA4): based on a mean grayscale value of each target cell area and a mean grayscale value of the blank area in each target image obtained at step (6IA3), calculating a first absorbance of each target cell using a formula: the first absorbance of each target cell=log (the mean grayscale value of the blank area/the mean grayscale value of each target cell area);
(6IA5): obtaining a mean value of the first absorbance based on the first absorbance of each target cell obtained at step (6IA4);
(6IA6): obtaining each target cell area and calculating a mean area of all target cells; and
(6IA7): calculating the first hemoglobin absorption coefficient using a formula: the first hemoglobin absorption coefficient=the mean value of the first absorbance÷the corpuscular hemoglobin per unit area of the target cell; or the first hemoglobin absorption coefficient=the mean value of the first absorbance÷(the corpuscular hemoglobin per liter of blood cell sample÷the red blood cell concentration÷the mean area of all target cells)=the mean value of the first absorbance×the red blood cell concentration×the mean area of all target cells÷the corpuscular hemoglobin per liter of blood cell sample.

2. The hemoglobin analysis method according to claim 1, wherein, at step (6B), each target cell in the target image is an independent single cell.

3. The hemoglobin analysis method according to claim 1, wherein, the first hemoglobin absorption coefficient is a constant value corresponding to a target sample to be tested, or a constant value corresponding to the target sample to be tested obtained by looking up a data table.

4. The hemoglobin analysis method according to claim 1, comprising the following step:
(6M): obtaining a mean corpuscular hemoglobin of the target cells based on summing up a total of the corpuscular hemoglobin of each target cell.

5. The hemoglobin analysis method according to claim 1, comprising the following step:
(6M2): outputting a histogram of the corpuscular hemoglobin of each target cell based on the corpuscular hemoglobin of each target cell; wherein the histogram is configured to count a hemoglobin distribution pattern of different target cells.

6. The hemoglobin analysis method according to claim 1, comprising the following step:
(6M3): obtaining a volume of each target cell and outputting a CH-CV scatter plot according to the volume of each target cell and the corpuscular hemoglobin of each target cell;
wherein the CH-CV scatter plot is configured to statistically analyze a hemoglobin distribution pattern of target cells with different volumes.

7. The hemoglobin analysis method according to claim 6, comprising the following step:
(6M4): displaying at least one CH indicator line and at least one CV indicator line on the CH-CV scatter plot.

8. The hemoglobin analysis method according to claim 4, comprising the following steps:
(6N): obtaining a mean corpuscular volume; and
(6P): calculating the mean corpuscular hemoglobin concentration using a formula: the mean corpuscular hemoglobin concentration=the mean corpuscular hemoglobin of target cells÷the mean corpuscular volume.

9. The hemoglobin analysis method according to claim 4, comprising the following steps:
(6Q): obtaining a red blood cell concentration; and
(6R): calculating a corpuscular hemoglobin per unit volume of blood using a formula: the corpuscular hemoglobin per unit volume of blood=the mean corpuscular hemoglobin of target cells×the red blood cell concentration.

10. The hemoglobin analysis method according to claim 1,
wherein the microscopically magnified digital image is obtained under illumination of a wide-spectrum illumination light source; and
the microscopically magnified digital image is an R/G/B three-channel microscopically magnified digital image containing at least three-color component information; the R/G/B three-channel are a red channel, a green channel, and a blue channel respectively.

11. The hemoglobin analysis method according to claim 1,
wherein the microscopically magnified digital image is obtained under illumination of a specific light source; and
the specific light source is a purple light source with a central wavelength of 418 nm; the microscopically magnified digital image is an R/G/B three-channel microscopically magnified digital image containing at least three-color component information.

12. The hemoglobin analysis method according to claim 11,
wherein, at step (6C), based on the mean grayscale value of the blue channel of the target cell area in the target image and the mean grayscale value of the blue channel of the blank area in the target image, calculating the first absorbance of the target cell using a formula: the first absorbance of the target cell=log (the mean grayscale value of the blue channel of the blank area/the mean grayscale value of the blue channel of the target cell area);
at step (6IA4), based on the mean grayscale value of the blue channel of each target cell area and the mean grayscale value of the blue channel of the blank area in each target image in the target image obtained at step (6IA3), calculating the first absorbance of each target cell using a formula: the first absorbance of each target cell=log (the mean grayscale value of the blue channel of the blank area/the mean grayscale value of the blue channel of the target cell area);
at step (7IA4): based on the mean grayscale value of the blue channel of each target cell area and the mean grayscale value of the blue channel of the blank area in each target image obtained at step (7IA3), calculating the first absorbance of each target cell using a formula: the first absorbance of each target cell=log (the mean grayscale value of the blue channel of the blank area/the mean grayscale value of the blue channel of each target cell area); and
at step (8DA4), based on the mean grayscale value of the blue channel of each target cell area and the mean grayscale value of the blue channel of the blank area in each target image obtained at step (8DA3), calculating the first absorbance of each target cell using a formula: the first absorbance of each target cell=log (the mean grayscale value of the blue channel of the blank area/the mean grayscale value of the blue channel of each target cell area).

13. The hemoglobin analysis method according to claim 10,
wherein, at step (6C), based on the mean grayscale value of any channel of the target cell area in the target image and the mean grayscale value of any channel of the blank area in the target image, calculating the first absorbance of the target cell using a formula: the first absorbance of the target cell=log (the mean grayscale value of any channel of the blank area/the mean grayscale value of any channel of the target cell area);
at step (6IA4), based on the mean grayscale value of any channel of the target cell area and the mean grayscale value of any channel of the blank area in the target image obtained at step (6IA3), calculating the first absorbance of each target cell using a formula: the first absorbance of each target cell=log (the mean grayscale value of any channel of the blank area/the mean grayscale value of any channel of the target cell area);
at step (7IA4), based on the mean grayscale value of any channel of each target cell area and the mean grayscale value of any channel of the blank area in the target image obtained at step (7IA3), calculating the first absorbance of each target cell using a formula: the first absorbance of each target cell=log (the mean grayscale value of any channel of the blank area/the mean grayscale value of any channel of the target cell area);
at step (8DA4), based on the mean grayscale value of any channel of each target cell area and the mean grayscale value of any channel of the blank area in the target image obtained at step (8DA3), calculating the first absorbance of each target cell using a formula: the first absorbance of each target cell=log (the mean grayscale value of any channel of the blank area/the mean grayscale value of any channel of the target cell area); and
any channel comprises a red channel, a green channel, and a blue channel.

14. A hemoglobin analysis method based on a microscopically magnified digital image, wherein the microscopically magnified digital image is obtained based on blood cells laid in a monolayer in a suspension; and the method comprises the following steps:
(6A): identifying multiple target cells in the microscopically magnified digital image;
(6B): selecting a target image corresponding to each target cell in the microscopically magnified digital image; wherein the target image comprises a target cell area and a blank area;
(6C): based on a mean grayscale value of the target cell area in the target image and a mean grayscale value of the blank area in the target image, calculating a first absorbance of the target cell using a formula: the first absorbance of the target cell=log (the mean grayscale value of the blank area in the target image/the mean grayscale value of the target cell area in the target image);
(7I): obtaining a known first corpuscular hemoglobin correction coefficient;
(7K): obtaining each target cell area in the microscopically magnified digital image;
(7J): calculating a target cell corpuscular hemoglobin using a formula: the target cell corpuscular hemoglobin=the first absorbance×each target cell area×the first corpuscular hemoglobin correction coefficient;
wherein the step (7I) of obtaining the first corpuscular hemoglobin correction coefficient comprises the following steps:
(7IA1): taking an amount of blood cell sample to be analyzed, and obtaining a corpuscular hemoglobin and a red blood cell concentration per liter of the blood cell sample using a hemoglobin analyzer;
(7IA2): taking a same amount of the blood cell sample to be analyzed as at step (7IA1), obtaining a cell suspension by pre-treating, injecting the cell suspension into an imaging target area; making a monolayer of blood cells in the suspension, and obtaining a microscopically magnified digital image of the monolayer of blood cells in the suspension;
(7IA3): selecting a target image corresponding to each target cell in the microscopically magnified digital image obtained at step (7IA2), wherein the corresponding target image comprises a target cell area and a blank area;
(7IA4): based on a mean grayscale value of each target cell area and a mean grayscale value of the blank area in each target image obtained at step (7IA3), calculating a first absorbance of each target cell using a formula: the first absorbance of each target cell=log (the mean grayscale value of the blank area in each target image/the mean grayscale value of each target cell area);
(7IA5): obtaining a mean value of the first absorbance based on the first absorbance of each target cell obtained at step (7IA4);
(7IA6): obtaining each target cell area and calculating a mean area of all target cells; and
(7IA7): calculating the first corpuscular hemoglobin correction coefficient using a formula: the first corpuscular hemoglobin correction coefficient=the corpuscular hemoglobin per liter of blood cell sample÷the red blood cell concentration÷the mean value of the first absorbance÷the mean area of all target cells.

15. The hemoglobin analysis method according to claim 14, wherein the first corpuscular hemoglobin correction coefficient is a constant value corresponding to the target sample to be tested, or a constant value corresponding to the target sample to be tested obtained by looking up a data table.

16. A hemoglobin analysis method based on a microscopically magnified digital image, wherein the microscopically magnified digital image is obtained based on blood cells laid in a monolayer in a suspension; and the method comprises:
(6A): identifying multiple target cells in the microscopically magnified digital image;
(6B): selecting a target image corresponding to each target cell in the microscopically magnified digital image; wherein the target image comprises a target cell area and a blank area;
(6C): based on a mean grayscale value of the target cell area in the target image and a mean grayscale value of the blank area in the target image, calculating a first absorbance of the target cell using a formula: the first absorbance of the target cell=log (the mean grayscale value of the blank area in the target image/the mean grayscale value of the target cell area in the target image);
(8D): obtaining a first hemoglobin concentration correction coefficient;
(8E): calculating a hemoglobin concentration of a single target cell using a formula: the hemoglobin concentration of a single target cell=the first absorbance×the first hemoglobin concentration correction coefficient;
wherein the step (8D) of obtaining the first hemoglobin concentration correction coefficient comprises the following steps:
(8DA1): taking an amount of blood cell sample to be analyzed; and obtaining a corpuscular hemoglobin, a red blood cell concentration per liter of the blood cell sample, and a mean corpuscular volume per liter of the blood cell sample using a hemoglobin analyzer;
(8DA2): taking a same amount of blood cell sample to be analyzed as at step (8DA1), obtaining a cell suspension by pre-treating, and injecting the cell suspension into an imaging target area; making a monolayer of blood cells in the suspension, and obtaining a microscopically magnified digital image of the monolayer of blood cells in the suspension;
(8DA3): selecting a target image corresponding to each target cell in the microscopically magnified digital image obtained at step (8DA2); wherein the target image comprises a target cell area and a blank area;
(8DA4): based on a mean grayscale value of each target cell area and a mean grayscale value of the blank area in each target image obtained at step (8DA3), calculating a first absorbance of each target cell using a formula: the first absorbance of each target cell=log (the mean grayscale value of the blank area/the mean grayscale value of each target cell area);
(8DA5): calculating a mean value of the first absorbance based on the first absorbance of each target cell obtained at step (8DA4); and
(8DA6): calculating a first hemoglobin concentration correction coefficient using a formula: the first hemoglobin concentration correction coefficient=the corpuscular hemoglobin per liter of blood cell sample÷the red blood cell concentration÷the mean corpuscular volume÷the mean value of the first absorbance.

17. The hemoglobin analysis method according to claim 16, wherein the first hemoglobin concentration correction coefficient is a constant value corresponding to the target sample to be tested, or a constant value corresponding to the target sample to be tested obtained by looking up a data table.

18. The hemoglobin analysis method according to claim 16, comprising the following step:
(6F): obtaining a hemoglobin concentration of all target cells in the microscopically magnified digital image and calculating the mean corpuscular hemoglobin concentration using a formula: the mean corpuscular hemoglobin concentration=Σ(CHGBs)÷the number of all target cells, wherein CHGBs represents the hemoglobin concentration of all target cells in the microscopically magnified digital image.

19. The hemoglobin analysis method according to claim 16, comprising the following steps:
(6G): identifying the target cells in the microscopically magnified digital image using AI algorithm, obtaining a single target cell area in the microscopically magnified digital image; and obtaining a known mean cell height; and
(6H): calculating a corpuscular hemoglobin of a single target cell using a formula: the corpuscular hemoglobin of a single target cell=the single target cell area xa hemoglobin concentration of a single target cell×the mean cell height.

20. A hemoglobin analysis method based on a microscopically magnified digital image, wherein the microscopically magnified digital image is obtained based on blood cells laid in a monolayer in a suspension, and the method comprises the following steps:
(9A): identifying a target cell area and a blank area in the microscopically magnified digital image; wherein the target cell area comprises a target cell area corresponding to a single target cell and/or a target cell area corresponding to multiple cells overlapped;
(9C): based on a mean grayscale value of the target cell area and a mean grayscale value of the blank area in the target image, calculating a first absorbance of the target cell using a formula: the first absorbance of the target cell=log (the mean grayscale value of the blank area/the mean grayscale value of the target cell area);
(9I): obtaining a first hemoglobin absorption coefficient;
(9J): calculating a corpuscular hemoglobin per unit area of the target cell area using a formula: the corpuscular hemoglobin per unit area of the target cell area=the first absorbance/the first hemoglobin absorption coefficient;
(9K): obtaining a total target cell area and the number of target cells corresponding to the target cell area in the microscopically magnified digital image;
(9L): calculating a corpuscular hemoglobin of each target cell using a formula: the corpuscular hemoglobin of each target cell=the total target cell area×the corpuscular hemoglobin per unit area of the target cell÷the number of target cells, or the corpuscular hemoglobin of each target cell=the total target cell area÷the number of target cells÷the first hemoglobin absorption coefficient×log (the mean grayscale value of the blank area/the mean grayscale value of the target cell area); and
wherein the step (9I) of obtaining the first hemoglobin absorption coefficient comprises the following steps:
(6IA1): taking an amount of blood cell sample to be analyzed, and obtaining a corpuscular hemoglobin and a red blood cell concentration per liter of the blood cell sample using a hemoglobin analyzer;
(6IA2): taking a same amount of the blood cell sample to be analyzed as at step (6IA1), obtaining a cell suspension by pre-treating, injecting the cell suspension into an imaging target area; making a monolayer of blood cells in the suspension, and obtaining a microscopically magnified digital image of the monolayer of blood cells in the suspension;
(6IA3): selecting a target image corresponding to each target cell in the microscopically magnified digital image obtained at step (6IA2), wherein the corresponding target image comprises a target cell area and a blank area;
(6IA4): based on a mean grayscale value of each target cell area and a mean grayscale value of the blank area in each target image obtained at step (6IA3), calculating a first absorbance of each target cell using a formula: the first absorbance of each target cell=log (the mean grayscale value of the blank area/the mean grayscale value of each target cell area);
(6IA5): obtaining a mean value of the first absorbance based on the first absorbance of each target cell obtained at step (6IA4);
(6IA6): obtaining each target cell area and calculating a mean area of all target cells; and
(6IA7): calculating the first hemoglobin absorption coefficient using a formula: the first hemoglobin absorption coefficient=the mean value of the first absorbance÷the corpuscular hemoglobin per unit area of the target cell; or the first hemoglobin absorption coefficient=the mean value of the first absorbance÷(the corpuscular hemoglobin per liter of blood cell sample÷the red blood cell concentration÷the mean area of all target cells)=the mean value of the first absorbance×the red blood cell concentration×the mean area of all target cells÷the corpuscular hemoglobin per liter of blood cell sample.

21. An electronic device, comprising: a memory, a processor, and a computer program stored in the memory and executable by the processor, wherein the processor is configured to execute the computer program to realize a hemoglobin analysis method based on a microscopically magnified digital image, the microscopically magnified digital image is obtained based on blood cells laid in a monolayer in a suspension, and the method comprises the following steps:
(6A): identifying multiple target cells in the microscopically magnified digital image;
(6B): selecting a target image corresponding to each target cell in the microscopically magnified digital image; wherein the target image comprises a target cell area and a blank area;
(6C): based on a mean grayscale value of the target cell area in the target image and a mean grayscale value of the blank area in the target image, calculating a first absorbance of the target cell using a formula: the first absorbance of the target cell=log (the mean grayscale value of the blank area in the target image/the mean grayscale value of the target cell area in the target image);
(6I): obtaining a first hemoglobin absorption coefficient;
(6J): calculating a corpuscular hemoglobin per unit area of the target cell using a formula:
the corpuscular hemoglobin per unit area of the target cell=the first absorbance of the target cell/the first hemoglobin absorption coefficient;
(6K): obtaining each target cell area in the microscopically magnified digital image; and
(6L): calculating a corpuscular hemoglobin of each target cell using a formula: the corpuscular hemoglobin of each target cell=each target cell area×the corpuscular hemoglobin per unit area of the target cell, or the corpuscular hemoglobin of each target cell=each target cell area/the first hemoglobin absorption coefficient×log (the mean grayscale value of the blank area in the target image/the mean grayscale value of the target cell area in the target image);
wherein the step (6I) of obtaining the first hemoglobin absorption coefficient comprises the following steps:
(6IA1): taking an amount of blood cell sample to be analyzed, and obtaining a corpuscular hemoglobin and a red blood cell concentration per liter of the blood cell sample using a hemoglobin analyzer;
(6IA2): taking a same amount of the blood cell sample to be analyzed as at step (6IA1), obtaining a cell suspension by pre-treating, injecting the cell suspension into an imaging target area; making a monolayer of blood cells in the suspension, and obtaining a microscopically magnified digital image of the monolayer of blood cells in the suspension;
(6IA3): selecting a target image corresponding to each target cell in the microscopically magnified digital image obtained at step (6IA2), wherein the corresponding target image comprises a target cell area and a blank area;
(6IA4): based on a mean grayscale value of each target cell area and a mean grayscale value of the blank area in each target image obtained at step (6IA3), calculating a first absorbance of each target cell using a formula: the first absorbance of each target cell=log (the mean grayscale value of the blank area/the mean grayscale value of each target cell area);
(6IA5): obtaining a mean value of the first absorbance based on the first absorbance of each target cell obtained at step (6IA4);
(6IA6): obtaining each target cell area and calculating a mean area of all target cells; and
(6IA7): calculating the first hemoglobin absorption coefficient using a formula: the first hemoglobin absorption coefficient=the mean value of the first absorbance÷the corpuscular hemoglobin per unit area of the target cell; or the first hemoglobin absorption coefficient=the mean value of the first absorbance÷(the corpuscular hemoglobin per liter of blood cell sample÷the red blood cell concentration÷the mean area of all target cells)=the mean value of the first absorbance×the red blood cell concentration×the mean area of all target cells÷the corpuscular hemoglobin per liter of blood cell sample.

22. A readable storage medium, storing a computer program, wherein the processor is configured to execute the computer program to realize a hemoglobin analysis method based on a microscopically magnified digital image, the microscopically magnified digital image is obtained based on blood cells laid in a monolayer in a suspension, and the method comprises the following steps:
(6A): identifying multiple target cells in the microscopically magnified digital image;
(6B): selecting a target image corresponding to each target cell in the microscopically magnified digital image; wherein the target image comprises a target cell area and a blank area;
(6C): based on a mean grayscale value of the target cell area in the target image and a mean grayscale value of the blank area in the target image, calculating a first absorbance of the target cell using a formula: the first absorbance of the target cell=log (the mean grayscale value of the blank area in the target image/the mean grayscale value of the target cell area in the target image);
(6I): obtaining a first hemoglobin absorption coefficient;
(6J): calculating a corpuscular hemoglobin per unit area of the target cell using a formula:
the corpuscular hemoglobin per unit area of the target cell=the first absorbance of the target cell/the first hemoglobin absorption coefficient;
(6K): obtaining each target cell area in the microscopically magnified digital image; and
(6L): calculating a corpuscular hemoglobin of each target cell using a formula: the corpuscular hemoglobin of each target cell=each target cell area×the corpuscular hemoglobin per unit area of the target cell, or the corpuscular hemoglobin of each target cell=each target cell area/the first hemoglobin absorption coefficient×log (the mean grayscale value of the blank area in the target image/the mean grayscale value of the target cell area in the target image);
wherein the step (6I) of obtaining the first hemoglobin absorption coefficient comprises the following steps:
(6IA1): taking an amount of blood cell sample to be analyzed, and obtaining a corpuscular hemoglobin and a red blood cell concentration per liter of the blood cell sample using a hemoglobin analyzer;
(6IA2): taking a same amount of the blood cell sample to be analyzed as at step (6IA1), obtaining a cell suspension by pre-treating, injecting the cell suspension into an imaging target area; making a monolayer of blood cells in the suspension, and obtaining a microscopically magnified digital image of the monolayer of blood cells in the suspension;
(6IA3): selecting a target image corresponding to each target cell in the microscopically magnified digital image obtained at step (6IA2), wherein the corresponding target image comprises a target cell area and a blank area;
(6IA4): based on a mean grayscale value of each target cell area and a mean grayscale value of the blank area in each target image obtained at step (6IA3), calculating a first absorbance of each target cell using a formula: the first absorbance of each target cell=log (the mean grayscale value of the blank area/the mean grayscale value of each target cell area);
(6IA5): obtaining a mean value of the first absorbance based on the first absorbance of each target cell obtained at step (6IA4);
(6IA6): obtaining each target cell area and calculating a mean area of all target cells; and
(6IA7): calculating the first hemoglobin absorption coefficient using a formula: the first hemoglobin absorption coefficient=the mean value of the first absorbance÷the corpuscular hemoglobin per unit area of the target cell; or the first hemoglobin absorption coefficient=the mean value of the first absorbance÷(the corpuscular hemoglobin per liter of blood cell sample÷the red blood cell concentration÷the mean area of all target cells)=the mean value of the first absorbance×the red blood cell concentration×the mean area of all target cells÷the corpuscular hemoglobin per liter of blood cell sample.
